(19) United States
(12) Patent Application Publication
(10) Pub. No.: US 2026/0335253 A1
(43) Pub. Date: Oct. 29, 2026

(54) COMPOSITIONS AND METHODS FOR AMELIORATION OF SYMPTOMS ASSOCIATED WITH CLEC16A DYSFUNCTION OR LOSS

(71) Applicant: THE CHILDREN'S HOSPITAL OF PHILADELPHIA, Philadelphia, PA (US)

(72) Inventors: Hakon HAKONARSON, Malvern, PA (US); Rahul PANDEY, Wallingford, PA (US); Marina BAKAY, Parkesburg, PA (US); Bryan STRENKOWSKI, Browns Mills, NJ (US)

(73) Assignee: THE CHILDREN'S HOSPITAL OF PHILADELPHIA, Philadelphia, PA (US)

(21) Appl. No.: 19/107,920

(22) PCT Filed: Sep. 7, 2023
(86) PCT No.: PCT/US2023/032170

§ 371 Date: Feb. 28, 2025

Related U.S. Application Data
(60) Provisional application No. 63/374,981, filed on Sep. 8, 2022.

(51) Int. Cl. A61K 31/10; A61K 31/437; A61K 31/4985; A61K 31/506; A61K 31/519; A61K 31/541; A61P 3/00

(52) U.S. Cl. CPC A61K 31/10; A61K 31/437; A61K 31/4985; A61K 31/506; A61K 31/519; A61K 31/541; A61P 3/00

(57) ABSTRACT

The present disclosure provides compositions and methods for the treatment of CLEC16A-associated phenotypes and disorders. The present disclosure further provides a method for treatment of CLEC-16A-associated degeneration of the thymus, spleen, inguinal white adipose tissue, and gonal white adipose tissue. Further, wherein a method for treatment of CLEC16A-associated symptoms in a subject in need thereof comprises diagnosing the subject and administering an effective amount of mitophagy enhancer.

### CROSS-REFERENCE TO RELATED APPLICATION

This application claims priority of US Provisional application number 63/374,981 filed Sep. 8, 2022, the entire contents being incorporated herein by reference as though set forth in full.

### FIELD OF THE INVENTION

The present invention relates the fields of the amelioration of symptoms associated with CLEC16A dysfunction or loss. More specifically, the invention provides agents useful for the treatment of autoimmune disorders, lipodystrophic disorders, and neurodegenerative disorders in patients in need thereof.

### BACKGROUND OF THE INVENTION

Several publications and patent documents are cited through the specification in order to describe the state of the art to which this invention pertains. Each of these citations is incorporated herein by reference as though set forth in full.

CLEC16A is implicated in multiple autoimmune diseases. Turning off CLEC16A in adult mice leads to dysregulated mitophagy, severe weight loss, robust autoimmune inflammatory responses, severe neurological symptoms with neuroinflammation and progressive neurodegeneration resembling spinocerebellar ataxia. Adult CLEC16A knockout mice (CLEC16A^{ΔUBC} mice) models represent a sensory ataxia with dystonia, immune and inflammatory components seen in some human cerebellar ataxias and sensory neuronopathies along with lipodystrophy.

The ongoing hypothesis that observed perturbations in mitophagy/autophagy resulting in observed phenotype can be rescued was tested, at least in part, by interventions targeting the mitophagy/autophagy, JAK-STAT pathway, including but not limited to SOCS1 and tofacitinib using a drug repurposing therapy approach. Treatment with a JAK/STAT pan-inhibitor (tofacitinib) partially rescued the phenotype and improved survival of CLEC16A KO mice by modulating ER stress, lipolysis, mitophagy and autophagy. However, aberrant, partial phenotypic features remained.

Given the large number of autoimmune, and other disorders correlated with CLEC16A loss or dysfunction, it is clear that new treatments and therapeutic agents which help ameliorate the effects of malfunctioning CLEC16A, and reduce associated symptoms, are urgently needed.

### SUMMARY OF THE INVENTION

In accordance with the present invention, a method for ameliorating at least one CLEC16A-associated symptom in a subject in need thereof, comprising administering an effective amount of at least one mitophagy enhancer, wherein said at least one symptom is selected from robust autoimmune inflammatory responses, severe weight loss, severe neurological symptoms, neuroinflammation, progressive neurodegeneration resembling spinocerebellar ataxia, and fat loss is provided. Also provided herein are methods for treating a CLEC16A-associated disorder in a subject in need thereof, comprising administering an effective amount of at least one mitophagy enhancer. In certain embodiments, the CLEC16A-associated disorder is selected from an autoimmune disorder, a lipodystrophic disorder, and a neurodegenerative disorder.

In certain embodiments of the invention, the at least one mitophagy enhancer enhances the clearance of mitochondria. Exemplary mitophagy enhancers are probucol, quercetin, or acipimox. In certain aspects, the methods further comprise administering one or more agents selected from a JAK-STAT inhibitor, an ER stress modulator, and a SOCS1 inhibitor. Exemplary JAK-STAT inhibitors are selected from tofacitinib, ruxolitinib, baricitinib, peficitinib, decernotiniba, filgotinib, solcitinibb, itacitinib, SHR0302, upadacitinib, and PF-04965842. Exemplary ER stress modulators are selected from Rapamycin, 4-phenylbutyric acid (4-PBA), trimethylamine N-oxide dehydrate (TMAO), dimethyl sulfoxide (DMSO), tauroursodeoxycholic acid (TUDCA), AMPK-activated protein kinase, 5′-aminoimidazole-4-carboxymide-1-β-d-2 ribofuranoside (AICAR), Glucagon-Like Peptide-1 (GLP-1), DPP4 inhibitors, and n-acetylcysteine (NAC). In certain embodiments, the methods further comprise administration of a PPARγ inhibitor.

In certain embodiments, the methods described herein rescue spleen atrophy and/or improves the organ weight ratio of Thymus, inguinal white adipose tissue (iWAT), and/or gonal white adipose tissue (gWAT) when compared to an untreated control. In certain aspects the treatment delays CLEC16A-associated symptom progression when compared to an untreated control.

Also provided herein are methods for treating CLEC16A-associated degeneration of the thymus, comprising administration of a mitophagy enhancer, thereby altering the weight ratio in thymus and ameliorating symptoms associated with degeneration of the thymus. Methods for treating CLEC16A-associated degeneration of spleen, comprising administration of a mitophagy enhancer, thereby altering the weight ratio in spleen, providing therapeutic benefit, and ameliorating symptoms associated with degeneration of the spleen, are also provided herein.

In another aspect of the invention, methods for treating CLEC16A-associated degeneration of iWAT, comprising administration of a mitophagy enhancer, thereby altering the weight ratio in iWAT and ameliorating symptoms associated with degeneration of the iWAT are provided. Also provided herein are methods for treating CLEC16A-associated degeneration of gWAT, comprising administration of a mitophagy enhancer, thereby altering the weight ratio in gWAT and ameliorating symptoms associated with degeneration of the gWAT.

Also provided herein are methods for treating CLEC16A-associated symptoms in a subject in need thereof, the method comprising, a) diagnosing the subject with a CLEC16A-associated disorder, and b) administering an effective amount of a mitophagy enhancer.

### BRIEF DESCRIPTION OF THE DRAWINGS

FIG. 1. Body weight in control±probucol and CLEC16A KO±probucol mice fed with standard chow.

FIGS. 2A-2L. Organ weight/body weight ratios for control±probucol and KO±probucol groups.

FIGS. 3A-3C. Probucol rescues response and disrupted mitophagy in KO mice splenic lysates. (FIG. 3A) Immunoblot analysis of spleen lysates depicting disrupted mitophagy and rescue by probucol. (FIG. 3B) Quantitation graph depicts expression levels of CLEC16A, P62, LC3I/II, PINK1, and Parkin from control±probucol and KO±probucol treated mice. (FIG. 3C) Schematic depicts probucol action in clearing defective mitochondria in Mitophagy.

FIG. 4. Probucol delays the phenotype progression in KO mice. Timeline to onset of disability (scores 1-4) in a cohort of control±probucol and KO±probucol mice after treating with tamoxifen for 4 successive days. Number of mice/group indicated on each graph; both ***P<0.001.

FIG. 5. Probucol partially rescues the lipodystrophic phenotype and improve the survival of CLEC16A KO mice. Representative dorsal and ventral dissection image depicting gross morphology and distribution of fat in control, KO and KO±probucol treated mice. Bottom panel shows amount of iWAT, BAT, and gWAT harvested from control, KO and KO±probucol (score 1, 2, and 3.5) mice.

FIG. 6A-6B. Quercetin attenuates the CLEC16a KO phenotype.

FIG. 7A-7C. Organ weight/body weight ratios for control±quercetin and KO±quercetin groups and control±acipimox and KO±acipimox groups.

FIG. 8A-8C. CLEC16A KO phenotype (FIG. 8A). Probucol mediated rescue in clearing late stage dysregulated mitophagy (FIG. 8B). Experimental design and Probucol dosage and administration over the course of the study (FIG. 8C)

FIG. 9. Late-stage mitophagy enhancer delays phenotype progression in a dose dependent manner.

### DETAILED DESCRIPTION OF THE INVENTION

Loss of CLEC16A results in several symptoms resulting in a specific phenotype. To address the phenotype feature, we chose to focus on the ultimate endpoint of mitophagy-clearance of damaged mitochondria to mitigate the negative consequences of mitochondrial damage. In a more tailored approach, using a drug repurposing therapy approach, we tested the mitophagy enhancer, probucol (lipid lowering drug) on our whole body inducible CLEC16A KO for rescue. Our findings provide evidence in support of targeting mitophagy alone or together with interventions at the JAK-STAT pathway results in phenotype rescue supporting such potential future therapeutic intervention in treating autoimmunity.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action. The terms “agent” and “test compound” denote a chemical compound, a mixture of chemical compounds, a biological macromolecule, or an extract made from biological materials such as bacteria, plants, fungi, or animal (particularly mammalian) cells or tissues.

It is also contemplated that the term “compound” or “compounds” refers to the compounds discussed herein and includes precursors and derivatives of the compounds, and pharmaceutically acceptable salts of the compounds, precursors, and derivatives. The invention also includes prodrugs of the compounds, pharmaceutical compositions including the compounds and a pharmaceutically acceptable carrier, and pharmaceutical compositions including prodrugs of the compounds and a pharmaceutically acceptable carrier.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

A “derivative” of a polypeptide, polynucleotide or fragments thereof means a sequence modified by varying the sequence of the construct, e.g. by manipulation of the nucleic acid encoding the protein or by altering the protein itself. “Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides.

The term “functional” as used herein implies that the nucleic or amino acid sequence is functional for the recited assay or purpose.

For purposes of the invention, “nucleic acid”, “nucleotide sequence” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

According to the present invention, an isolated or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “delivery” as used herein refers to the introduction of foreign molecule (i.e., miRNA containing nanoparticle) into cells. The term “administration” as used herein means the introduction of a foreign molecule into a cell. The term is intended to be synonymous with the term “delivery”.

As used herein, the phrase “effective amount” of a compound or pharmaceutical composition refers to an amount sufficient to modulate symptoms associated with aberrant CLEC16A expression in an animal, especially a human, including without limitation mitigation of the negative health consequences associated with mitochondrial damage and or reducing such consequences by prophylactic administration prior to the onset of symptoms.

Determination of an effective amount of the compound(s) for a particular use and mode of administration is well within the capabilities of those skilled in the art. Effective dosages may be estimated initially from in vitro activity and metabolism assays. For example, an initial dosage of compound for use in animals may be formulated to achieve a specific circulating blood or serum concentration of the metabolite active compound. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active metabolite compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective local dosages without undue experimentation.

Generally, the initial therapeutically effective amount of a compound described herein or a pharmaceutically acceptable salt thereof that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of the compound or a pharmaceutically acceptable salt thereof. In another embodiment, such dosage forms contain from about 50 mg to about 500 mg of the compound or a pharmaceutically acceptable salt thereof. In yet another embodiment, such dosage forms contain from about 25 mg to about 200 mg of the compound or a pharmaceutically acceptable salt thereof. In still another embodiment, such dosage forms contain from about 10 mg to about 100 mg of the compound or a pharmaceutically acceptable salt thereof. In a further embodiment, such dosage forms contain from about 5 mg to about 50 mg of the compound or a pharmaceutically acceptable salt thereof.

The phase “CLEC16A associated immune disorders” includes autoimmune lipody strophic and neurodegenerative disorders. Examples of such disorders include, without limitation, AD: Alzheimer's Disease; ADPD: Alzheimer's Disease and Parkinsons's Disease; AMDF: Ataxia, Myoclonus and Deafness, CIPO: Chronic Intestinal Pseudoobstruction with myopathy and Opthalmoplegia; CPEO: Chronic Progressive External Opthalmoplegia; DEAF: Maternally inherited DEAFness or aminoglycoside-induced Deafnness; DEMCHO: Dementia and Chorea; DMDF: Diabetes Mellitus & Deafness; Exercise Intolerance; ESOC: Epilepsy, Strokes, Optic atrophy, & 30 Cognitive decline; FBSN: Familial Bilateral Striatal Necrosis; FICP: Fatal Infantile Cardiomyopathy Plus, a MELAS-associated cardiomyopathy; GER: Gastrointestinal Reflux; KSS Kearns Sayre Syndrome LDYT: Leber's hereditary optic neuropathy and Dystonia; LHON: Leber Hereditary Optic Neuropathy; LIMM: Lethal Infantile Mitochondrial Myopathy; MDM: Myopathy and Diabetes Mellitus; MELAS: Mitochondrial Encephalomyopathy, Lactic Acidosis, and Stroke-like episodes; MEPR: Myoclonic Epilepsy and Psychomotor Regression; MERME:5 MERRF/MELAS overlap disease; MERRF: Myoclonic Epilepsy and Ragged Red Muscle Fibers; MHCM: Maternally Inherited Hypertrophic CardioMyopathy; MICM: Maternally Inherited Cardiomyopathy; MILS: Maternally Inherited Leigh Syndrome; Mitochondrial Encephalocardiomyopathy; Mitochondrial Encephalomyopathy; MM: Mitochondrial Myopathy; MMC: Maternal Myopathy and Cardiomyopathy; Multisystem Mitochondrial Disorder (myopathy, encephalopathy, blindness, hearing loss, peripheral neuropathy); NARP: Neurogenic muscle weakness, Ataxia, and Retinitis Pigmentosa; alternate phenotype at this locus is reported as Leigh Disease; NIDDM: Non-Insulin Dependent Diabetes Mellitus; PEM: Progressive Encephalopathy; PME: Progressive Myoclonus Epilepsy; RTT: Rett Syndrome; SIDS: Sudden Infant Death Syndrome, multiple sclerosis, primary adrenal insufficiency, Crohn's disease, primary biliary cirrhosis, juvenile idiopathic arthritis, rheumatoid arthritis, and alopecia areata, uveitis, and, lupus.

The phrase “CLEC16A associated phenotype” or “CLEC16A associated symptoms” includes at least one symptom from any CLEC16A disorder. Patients may experience a CLEC16A associated phenotype without being diagnosed with a CLEC16A associated immune disorder. Examples of CLEC16A associated phenotype include, without limitation, robust autoimmune inflammatory responses, severe weight loss, severe neurological symptoms, neuroinflammation, progressive neurodegeneration resembling spinocerebellar ataxia, and fat loss.

“Sample” or “patient sample” or “biological sample” generally refers to a sample which may be tested for a particular molecule, such as a marker described hereinbelow. Samples may include but are not limited to cells, body fluids, including blood, serum, plasma, cerebral spinal fluid, urine, saliva, tears, pleural fluid and the like.

The terms “agent” and “compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, and peptide/DNA complexes. Agents and compounds may also be referred to as “test agents” or “test compounds” which are evaluated for potential biological activity by inclusion in screening assays described herein below.

An “antilipidemic agent” or “antilipemic agent” refers to a compound that lowers levels of any or all lipids or lipoproteins, including fats, cholesterol or triglycerides, in the blood. In certain embodiments, the antilipidemic agent is administered to a patient with elevated levels of any or all lipids or lipoproteins. Certain antilipidemic agents inhibit cholesterol synthesis and/or delay cholesterol absorption. In certain embodiments, the antilipidemic agents inhibit oxidation of cholesterol in LDLs, and slows the formation of foam cells. Such inhibitors are known in the art and include, without limitation, siRNA molecules, peptide mimetics and small molecules. Examples of antilipidemic agents include, without limitation, probucol, Atorvastatin, Fluvastatin, Lovastatin, Pitavastatin, Pravastatin, Rosuvastatin, Simvastatin.

Mitophagy refers to a mitochondrial quality control mechanism that enables the degradation of damaged and superfluous mitochondria, thereby preventing mitochondrial dysfunction. A “mitophagy enhancer” refers to a compound that increases the degradation of damaged and superfluous mitochondria. In certain embodiments, an mitophagy enhancer is also an antilipidemic agent. In certain embodiments, the mitophagy enhancer is directed to the ultimate endpoint of mitophagy-clearance of damaged mitochondria. Such inhibitors are known in the art and include, without limitation, siRNA molecules, peptide mimetics and small molecules. Examples of mitophagy enhancers that enhance the clearance of damaged mitochondria include, without limitation, probucol, quercetin and acipimox.

Inhibitors of the JAK-STAT pathway also have utility in the present invention. In certain embodiments, the inhibitor is a JAK-STAT pan-inhibitor. Such inhibitors are known in the art and include, without limitation, siRNA molecules, peptide mimetics and small molecules. These include without limitation tofacitinib, ruxolitinib, baricitinib, peficitinib, decernotiniba, filgotinib, solcitinibb, itacitinib, SHR0302, upadacitinib, and PF-04965842.

The phrase “ER stress modulators” refers to a compound that increases or decreases defective unfolding of endoplastic reticulum folding. Such inhibitors are known in the art and include, without limitation, siRNA molecules, peptide mimetics and small molecules. These include without limitation mTOR inhibitors, Rapamycin, 4-phenylbutyric acid (4-PBA), trimethylamine N-oxide dehydrate (TMAO), dimethyl sulfoxide (DMSO), tauroursodeoxycholic acid (TUDCA), AMPK-activated protein kinase, 5′-aminoimidazole-4-carboxymide-1-β-d-ribofuranoside (AICAR), Glucagon-Like Peptide-1 (GLP-1), DPP4 inhibitors, and n-acetylcysteine (NAC). Additional modulators are known to those skilled in the art, See, e.g., Jung T W, Choi K M. Pharmacological Modulators of Endoplasmic Reticulum Stress in Metabolic Diseases. Int J Mol Sci. 2016 February 1;17(2):192. Doi: 10.3390/ijms 17020192. PMID:26840310; PMCID: PMC4783926. (Incorporated herein by reference).

Inhibitors of SOCS1 also have utility in the present invention. Such inhibitors are known in the art and include, without limitation, siRNA molecules, peptide mimetics and small molecules.

Modulators of the PPAR pathway including PPARa and PPARy also utility in the present invention. Such inhibitors are known in the art and include, without limitation, siRNA molecules, peptide mimetics and small molecules, such as fenofibrate.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

An “inhibitor” (interchangeably termed “antagonist”) of a polypeptide of interest is an agent that interferes with activation or function of the polypeptide of interest, e.g., partially or fully blocks, inhibits, or neutralizes a biological activity mediated by a polypeptide of interest. For example, an antagonist of polypeptide X may refer to any molecule that partially or fully blocks, inhibits, or neutralizes a biological activity mediated by polypeptide X. Examples of inhibitors include antibodies; ligand antibodies; small molecule antagonists; antisense and inhibitory RNA (e.g., siRNA) molecules. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

As used herein, the terms “modulate”, “modulation” or “modulation” refer to changing the rate at which a particular process occurs, inhibiting a particular process, reversing a particular process, and/or preventing the initiation of a particular process. The term “modulate” as used herein refers to increasing/promoting or decreasing/inhibiting a particular cellular, biological or signaling function associated with the normal activities of CLEC16A molecules described herein. For example, the term modulate refers to the ability of a test compound or test agent to interfere clearance of damaged mitochondria, thereby rescuing the CLEC16A phenotype.

The phrase “in need of treatment” as used herein refers to a judgment made by a caregiver (e.g., physician, nurse, nurse practitioner, or individual in the case of humans; veterinarian in the case of animals, including non-human mammals) that a subject requires or will benefit from treatment. This judgment is made based on a variety of factors that are in the realm of a care givers expertise, but that includes the knowledge that the subject is ill, or will be ill, as the result of a condition that is treatable by the disclosed compounds.

By “treatment” and “treating” is meant the medical management of a subject with the intent to cure, ameliorate, or stabilize, a pathological condition or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. It is understood that treatment, while intended to cure, ameliorate, or stabilize, a disease, pathological condition, or disorder, need not actually result in the cure, ameliorization, and stabilization. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

### Pharmaceutical Therapies

The elucidation of the role played by the CLEC16A facilitates the development of pharmaceutical compositions useful for treatment and diagnosis of phenotypes associated with aberrant CLEC16A function. These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

The compounds described herein can be formulated for enteral, parenteral, topical, or systemic administration. The compounds can be combined with one or more pharmaceutically acceptable carriers and/or excipients that are considered safe and effective and may be administered to an individual without causing undesirable biological side effects or unwanted interactions. The carrier is all components present in the pharmaceutical formulation other than the active ingredient or ingredients. Typical carriers and conventional methods of preparing pharmaceutical compositions that can be used in conjunction with the preparation of formulations of the compounds are known by those skilled in the art. These compositions can take the form of solutions, suspensions, emulsion, tablets, pills, capsules, powders, sustained-release formulations and the like.

Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, for example to a diluent, adjuvant, excipient, auxilliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A pharmaceutical composition of the present invention can be administered by any suitable route, for example, by injection, by oral, pulmonary, nasal or other forms of administration. In general, pharmaceutical compositions contemplated to be within the scope of the invention, comprise, inter alia, pharmaceutically acceptable diluents, preservatives, solubilizers, emulsifiers, adjuvants and/or carriers. Such compositions can include diluents of various buffer content (e.g., Tris HCl, acetate, phosphate), pH and ionic strength; additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435 1712 which are herein incorporated by reference. A pharmaceutical composition of the present invention can be prepared, for example, in liquid form, or can be in dried powder, such as lyophilized form. Particular methods of administering such compositions are described infra.

In yet another embodiment, a pharmaceutical composition of the present invention can be delivered in a controlled release system, such as using an intravenous infusion, an implantable osmotic pump, a transdermal patch, liposomes, or other modes of administration. In a particular embodiment, a pump may be used [see Langer, supra; Sefton, CRC Crit. Ref. Biomed. Eng. 14:201(1987); Buchwald et al., Surgery 88:507(1980); Saudek et al., N. Engl. J. Med. 321:574 (1989)]. In another embodiment, polymeric materials can be used [see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. 23:61(1983); see also Levy et al., Science 228:190(1985); During et al., Ann. Neurol. 25:351(1989); Howard et al., J. Neurosurg. 71:105(1989)]. In yet another embodiment, a controlled release system can be placed in proximity of the target tissues of the animal, thus requiring only a fraction of the systemic dose [see, e.g., Goodson, in Medical Applications of Controlled Release, supra, vol. 2, pp. 115 138(1984)]. Other controlled release systems are discussed in the review by Langer [Science 249:1527 1533(1990)].

### Methods Of Treatment

As discussed above the invention also includes methods for treating at CLEC16A-associated symptoms and disorders. CLEC16A-associated disorders include without limitation autoimmune disorders, lipodystrophic disorders and neurodegenerative disorders. An exemplary method entails administering a pharmaceutically effective amount of a mitophagy enhancer to a subject in need thereof. In certain embodiments, treatment can also include administration of a JAK-STAT inhibitor, an ER stress modulator, and/or a SOCS1 inhibitor.

In certain embodiments the method of treatment ameliorates symptoms associated with CLEC16A-associated symptoms and disorders. Symptoms can vary according to the type of CLEC16A-associated phenotype or disorder. In certain embodiments, symptoms of CLEC16A-associated phenotypes and disorders include without limitation, robust autoimmune inflammatory responses, severe weight loss, severe neurological symptoms, neuroinflammation, progressive neurodegeneration resembling spinocerebellar ataxia, and fat loss.

The following examples are provided to illustrate certain embodiments of the invention. They are not intended to limit the invention in any way.

### Example I

### Antilipidemic Agent Attenuates the Fat and Weight Loss of CLEC16A KO Mice

Turning off CLEC16A in adult mice leads to dysregulated mitophagy, robust autoimmune inflammatory responses[1], severe weight loss[2], severe neurological symptoms with neuroinflammation and progressive neurodegeneration resembling spinocerebellar ataxia[3]. Clearance of damaged mitochondria is a prerequisite for the survival of dopaminergic neurons, the cell type whose loss is responsible for the observed phenotype in CLEC16A KO mice. Current treatment options for vast majority of neurodegenerative disorders merely aim to modify symptoms and have no impact on the progression of the diseases; thus, a disease-modifying treatment remains a major unmet need. Therefore, repurposing drugs offers the opportunity to accelerate the clinical trials pipeline, given the presence of pre-existing pharmacological information about candidate compounds, to efficiently meet the unmet need in modulating neurodegeneration.

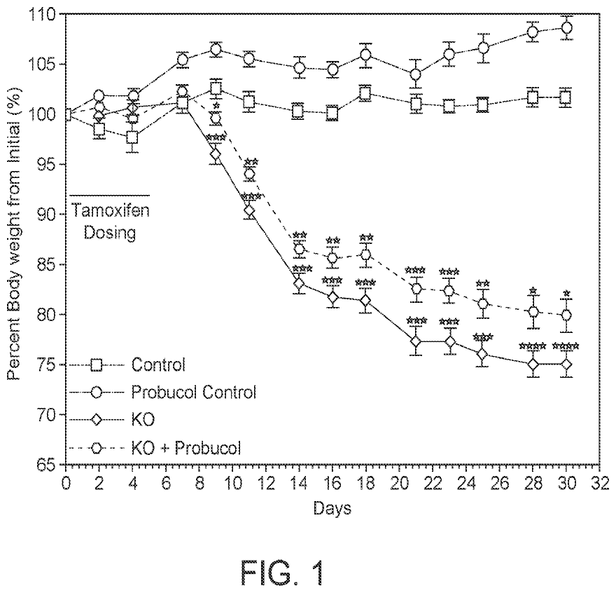

In light of previously published results and reported dysregulated mitophagy resulting in accumulation of defective mitochondria, we treated CLEC16A KO mice with probucol. Probucol is a lipid lowering drug and is characterized as an effective mitophagy enhancer in the literature[4]. Probucol is known to function downstream of PINK and parkin and modulates ultimate endpoint of mitophagy clearance of damaged mitochondria. If this step is improved, then the negative consequences of mitochondrial damage in dopaminergic neurons can be mitigated, resulting in rescue of the sensory neurodegeneration resembling spinocerebellar ataxia in KO mice. The CLEC16A KO mice exhibit significant reduction in body weight as early as day 9 compared to both controls. The decrease becomes more significant over the course of the study (FIG. 1). As anticipated probucol treatment significantly attenuated weight loss and improved the survival of CLEC16A KO mice (FIG. 1). Probucol treated control mice remained healthy and also gaincd significant weight through-out the study in comparison to control.

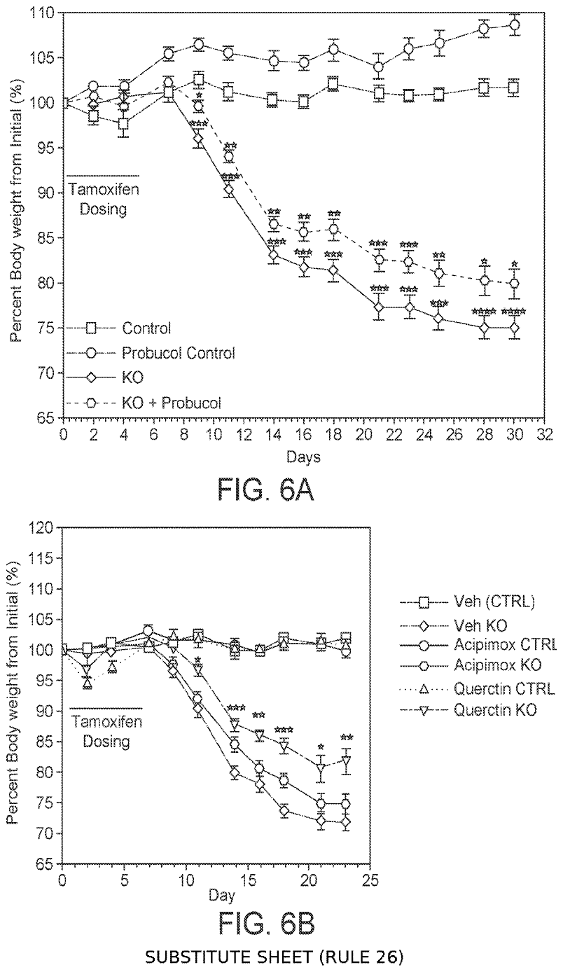

Additionally, we treated CLEC16A KO mice with quercetin and acipimox. The CLEC16A KO mice exhibit significant reduction in body weight as early as day 9 compared to both controls. The decrease becomes more significant over the course of the study (FIG. 6). As anticipated quercetin and acipimox treatment significantly attenuated weight loss and improved the survival of CLEC16A KO mice (FIG. 6). Quercetin and acipimox treated control mice remained healthy and also gained significant weight through-out the study in comparison to control.

### Example II

### Antilipidemic Agent Probucol Rescues Atrophy of Spleen, Thymus, Inguinal White Adipose Tissue (iWAT), Brown Adipose Tissue (BAT) and Gonal White Adipose Tissue (gWAT)

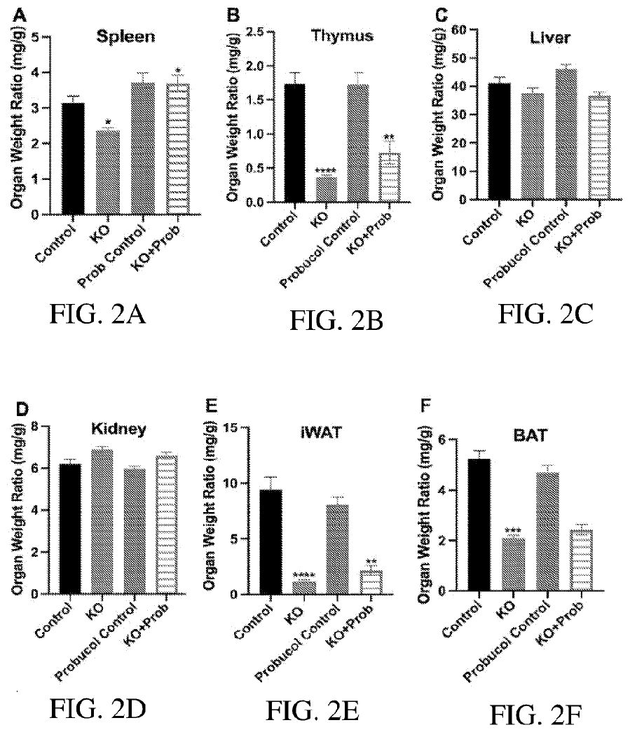

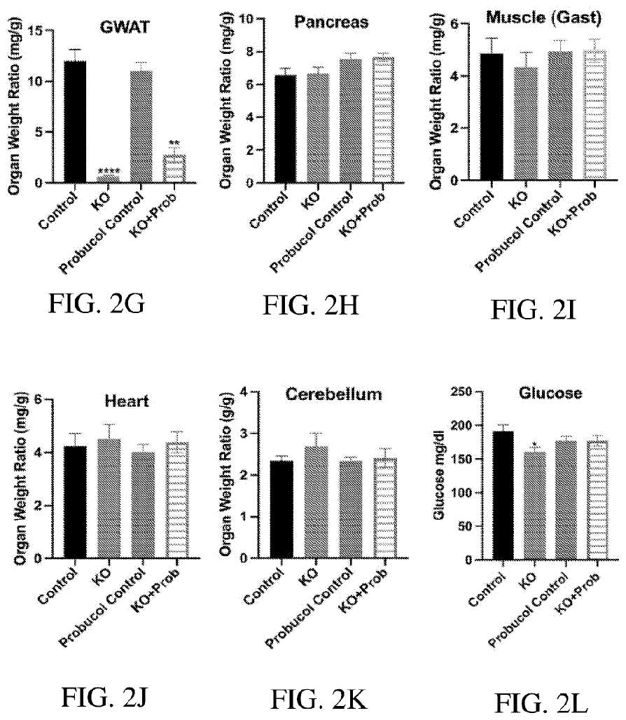

Spleen, thymus and white adipose atrophy is inducible in the CLEC16A knockout mice we created. Herein we evaluate the effect of probucol on organ weights of our whole body inducible CLEC16A knockout mice. FIG. 2 depicts the ratio graphs of spleen (FIG. 2A), thymus (FIG. 2B), liver (FIG. 2C), kidney (FIG. 2D) inguinal white adipose tissue (iWAT) (FIG. 2E), brown adipose tissue BAT (FIG. 2F), gonadal white adipose tissue gWAT (FIG. 2G), pancreas (FIG. 2H), muscle (FIG. 2I), heart (FIG. 2J), cerebellum (FIG. 2K), and glucose (FIG. 2L). In addition to weight loss and severe atrophy of the spleen (FIG. 2A), thymus (FIG. 2B) and inguinal white adipose tissue (IWAT) (FIG. 2E), we report severe atrophy of brown adipose tissue (BAT) (FIG. 2F) and gonadal white adipose tissue (gWAT) (FIG. 2G). KO mice exhibit significant reduction in glucose (FIG. 2L).

Treatment with probucol rescues the spleen atrophy (FIG. 2A). Probucol significantly improved the organ weight ratio of thymus (FIG. 2B), iWAT (FIG. 2E) and gWAT (FIG. 2G). The CLEC16A KO mice exhibited significant decrease in spleen (FIG. 2A), thymus (FIG. 2B), inguinal white adipose tissue (iWAT) (FIG. 2E), brown adipose tissue (BAT) (FIG. 2F) and gonadal white adipose tissue (gWAT)(FIG. 2G) weight and body weight ratios compared to controls over the course of the study.

The CLEC16A KO spleen, thymus, iWAT, BAT and gWAT weight/body weight ratio became significantly reduced in comparison to controls (FIG. 2). Liver, kidney, pancreas, muscle, heart, cerebellum showed no significant change in comparison to control littermates. Control tamoxifen and probucol control littermates showed no adverse effect on organ weight or their ratios over the course of the study. As anticipated, treatment with probucol significantly rescues the spleen atrophy. Probucol significantly improved the organ weight ratio of thymus, iWAT and gWAT. Mitophagy enhancer, probucol overall had a significant rescue effect on the organ weights of our whole body inducible CLEC16A knockout mice.

### Example III

### Antilipidemic Agents Quercetin and Acipimox Rescue Atrophy of Spleen, Thymus, Inguinal White Adipose Tissue (iWAT), Brown Adipose Tissue (BAT) and Gonal White Adipose Tissue (gWAT)

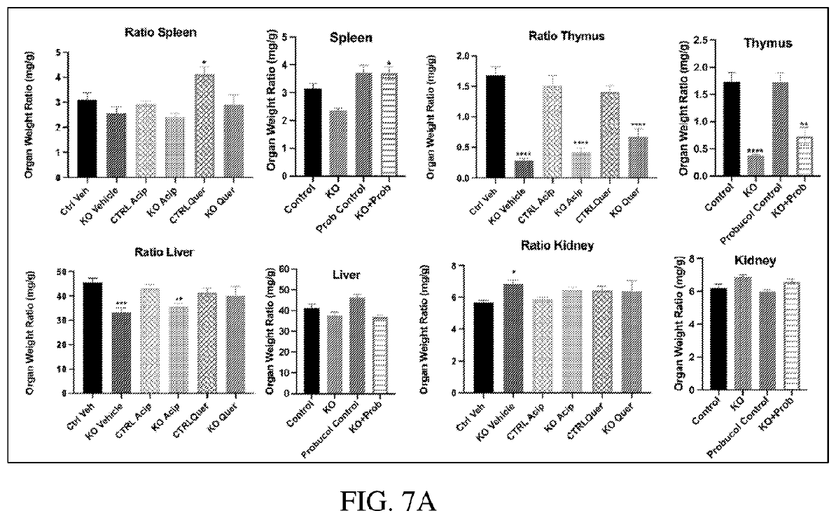

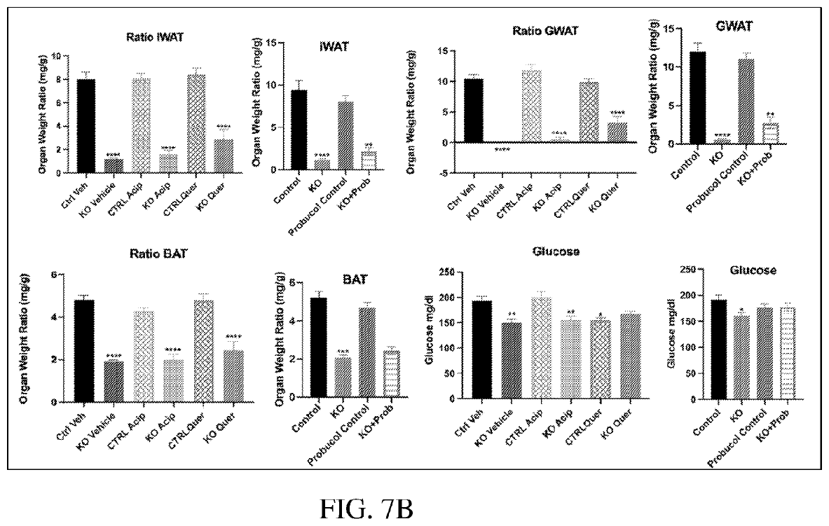

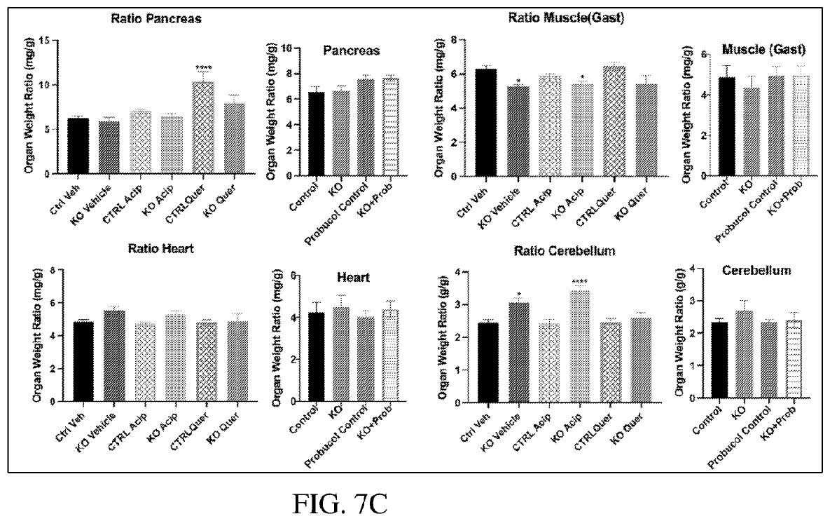

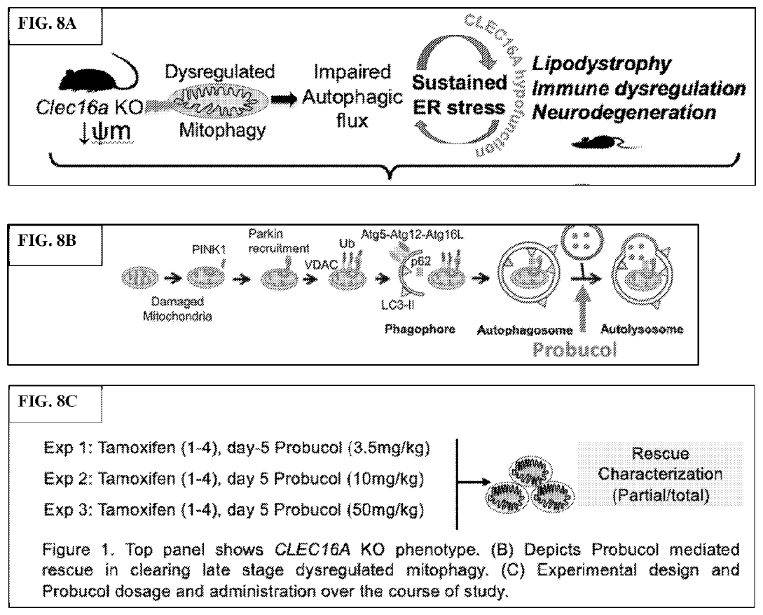

Spleen, thymus and white adipose atrophy is inducible in the CLEC16A knockout mice we created. Herein we evaluate the effect of quercetin or acipimox on organ weights of our whole body inducible CLEC16A knockout mice. FIG. 7 depicts the ratio graphs of spleen (FIG. 7A), thymus (FIG. 7A), liver (FIG. 7A), kidney (FIG. 7A) inguinal white adipose tissue (iWAT) (FIG. 7B), brown adipose tissue BAT (FIG. 7B), gonadal white adipose tissue gWAT (FIG. 7B), pancreas (FIG. 7C), muscle (FIG. 7C), heart (FIG. 7C), cerebellum (FIG. 7C), and glucose (FIG. 7B). In addition to weight loss and severe atrophy of the spleen, thymus, and inguinal white adipose tissue (IWAT), we report severe atrophy of brown adipose tissue (BAT) and gonadal white adipose tissue (gWAT). KO mice exhibit significant reduction in glucose.

Treatment with quercetin or acipimox rescues the spleen atrophy. Quercetin or acipimox significantly improved the organ weight ratio of thymus, iWAT and gWAT. The CLEC16A KO mice exhibited significant decrease in spleen, thymus, inguinal white adipose tissue (iWAT), brown adipose tissue (BAT) and gonadal white adipose tissue (gWAT) weight and body weight ratios compared to controls over the course of the study.

The CLEC16A KO spleen, thymus, iWAT, BAT and gWAT weight/body weight ratio became significantly reduced in comparison to controls (FIG. 7). Liver, kidney, pancreas, muscle, heart, cerebellum showed no significant change in comparison to control littermates. Control quercetin or acipimox control littermates showed no adverse effect on organ weight or their ratios over the course of the study. As anticipated, treatment with quercetin or acipimox significantly rescues the spleen atrophy. Quercetin or acipimox significantly improved the organ weight ratio of thymus, iWAT and gWAT. Mitophagy enhancer, quercetin or acipimox overall had a significant rescue effect on the organ weights of our whole body inducible CLEC16A knockout mice.

### Example IV

### Antilipidemic Agent Rescues the PINK-Parkin Dependent Dysregulated Mitophagy in Our Whole Body Inducible CLEC16A Knockout Mice

Incomplete mitophagy CLEC16A knockout disrupts Nrdp1/PINK/Parkin dependent mitophagy pathway in-vivo. This results in aggregation of fragmented mitochondria in splenic immune cells and predisposes mice to a cascade of altered signaling functions resulting in pathogenic inflammation, lipodystrophy, and sensory neurodegeneration neurodegeneration resembling spinocerebellar ataxia [2, 3, 5].

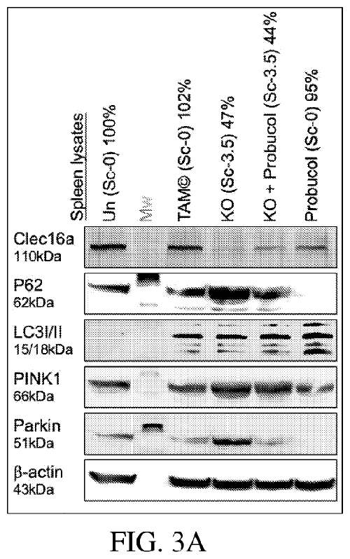

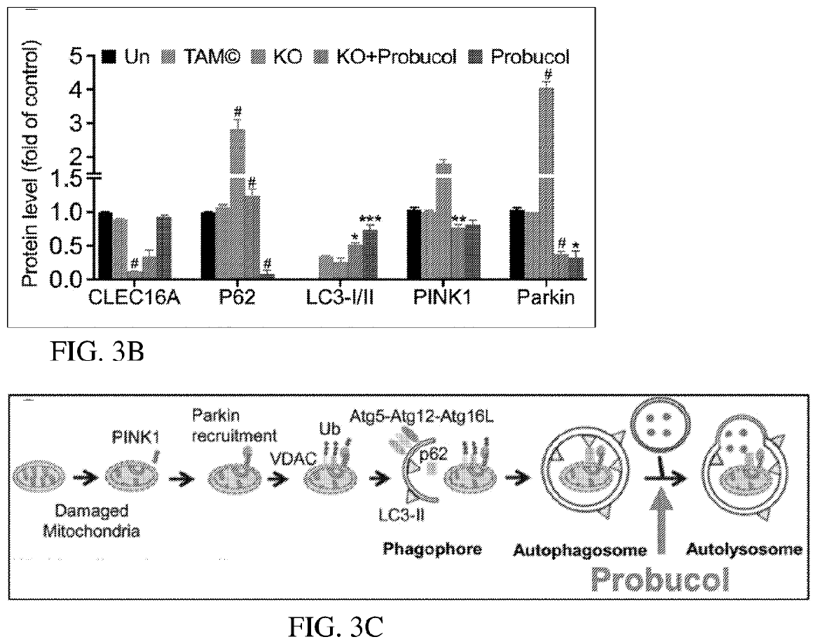

We next evaluated whole splenocyte lysates from control (Untreated/Vehicle-tamoxifen)±probucol and CLEC16A KO±probucol treated mice in an immunoblot analysis for possible mitophagy/autophagy signaling defects and rescue mediating by probucol (FIG. 3). Representative immune blot images depicting Nrdp1/PINK1/Parkin dependent incomplete mitophagy in CLEC16A KO mice spleen lysates are shown in FIG. 3A. As anticipated, CLEC16A KO mice splenocyte lysates showed increased expression of PINK1 and Parkin. Untreated and Vehicle-tamoxifen treated mice splenocyte lysates show similar detectable levels of PINK, Parkin and P62. The significant accumulation of p62 and reduced LC3-II expression in CLEC16A KO compared to both controls indicates disrupted mitophagy. Additionally, P62 is degraded by autophagy and inhibition of autophagy increases its abundance.

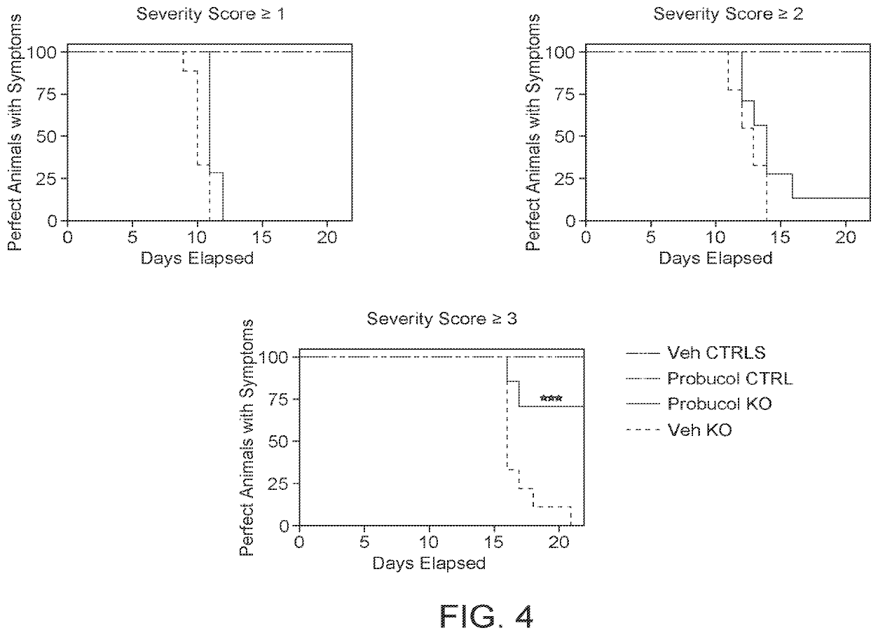

Probucol treated KO mice show significant decrease in levels of P62, PINK, Parkin and increase in LC3II. This indicates successful rescue of disrupted mitophagy. Control probucol treated mice exhibit further enhancement of basal mitophagy/autophagy. Percentages in FIG. 3 represent the level of CLEC16A left after excision. Untreated mice and mice treated with tamoxifen were scored based on their disability. (FIG. 4) CLECIA KO±probucol mice had a score of 3.5 indicating severe disability and phenotype. Probucol control mice had a score of 0 indicating no disability. Thus, probucol 1) functions downstream of PINK and parkin, and 2) modulates ultimate endpoint of mitophagy clearance of damaged mitochondria, and 3) improves overall survival of KO mice. Quantitation graph depicts expression levels of CLEC16A, p62, LC3I/II, PINK1 and Parkin from control±probucol and KO±probucol treated mice normalized to β-actin. Data expressed as mean±SE of three independent experiments. (FIG. 4)

### Example V

### Antilipidemic Agent Delays Phenotype Progression, Rescues Fat Loss, and Delays Lipodystrophy Progression

The timeline to onset of disability (scores 1-4) was determined in a cohort of control±probucol and KO±probucol mice after treating mice with tamoxifen for 4 successive days (FIG. 4). 10 mice/group were used. Both sexes were used in the study. Young adult UBC-Cre-ER-CLEC16a^{loxP/loxP} mice were treated with tamoxifen for four successive days to generate adult CLEC16A knockout (CLEC16A^{ΔUBC}) mice. Prior to treatment, CLEC16A^{loxP/loxP} and UBC-Cre-ERT2-Clec16a-^{loxP/loxP} were indistinguishable.

Approximately 8 days after tamoxifen induction, UBC-Cre-ERT2-CLEC16A^{loxP/loxP} (CLEC16A^{ΔUBC}) mice displayed weight loss that advanced quickly over days [2]. Tremors and other mild neurological behaviors (disability score=1; FIG. 4) started after approximately 10 days and progressed rapidly. About 60% of these mice exhibited the highest level of disability with dystonic postures after 25 days (disability score=4; FIG. 4).

The survival curve depicts severity of CLEC16A-associated phenotype. KO mice with a severity score of 3.5 exhibit the highest level of disability with dystonic postures after 25 days and fail to survive. Probucol treatment delays the progression of phenotype in KO mice. Only 25% of probucol treated KO mice reach severity score greater than 3 (FIG. 4). Thus, probucol functions downstream of PINK and parkin and modulates ultimate endpoint of mitophagy clearance of damaged mitochondria and improves survival of CLEC16A KO mice.

CLEC16A KO mice display extensive loss of body fat despite no decrease in food intake, together with compromised mitophagy and induction of systemic inflammatory response involving multiple cytokines/chemokines via inflammatory and classical lipolytic pathways [2].

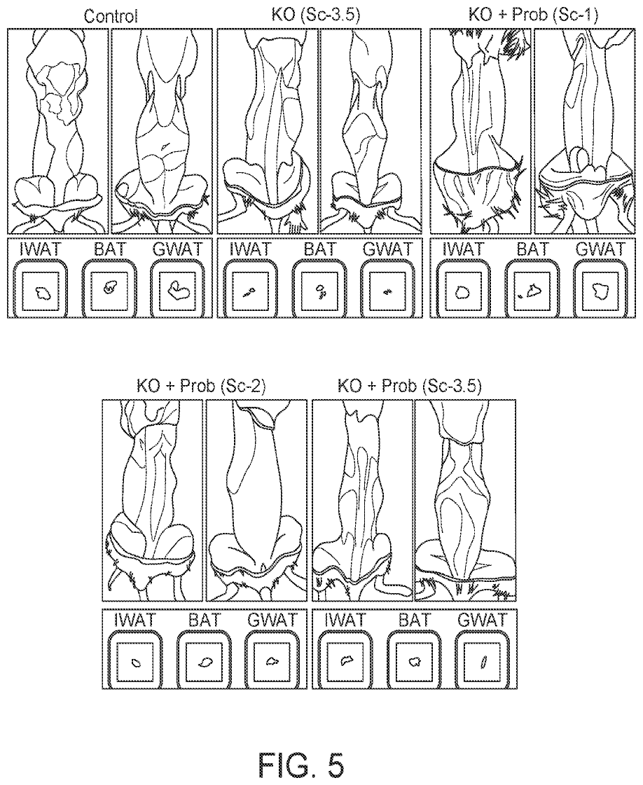

We next evaluated effect of probucol on fat loss and rescue of the lipodystrophic phenotype. Control, KO and KO±probucol mice (score 1, 2 and 3.5) were dissected and evaluated for fat distribution. We harvested inguinal WAT (iWAT), brown WAT (BAT), and gonadal WAT (gWAT) (FIG. 5). In comparison to control mice, KO mice had no inguinal and gonadal fat. Brown WAT also showed significant reduction. Treatment with probucol attenuates this fat loss. Probucol treated KO mice with severity scores of 1, 2 and 3 showed significant amount of iWAT, BAT and gWAT left in comparison to KO mice (Score-3.5). Thus, probucol rescues fat loss and delays lipodystrophy progression in KO mice over the length of the study. Probucol may facilitate more efficient transfer of lipid species or proteins for mitophagy steps downstream of Parkin, such as autophagosome expansion facilitating clearance of accumulated defective mitochondria. Robust lipid droplet expansion occurs following mitochondrial damage. A recent study has further found increased lipid droplet accumulation in the dopaminergic neurons of Parkinson's disease (PD) patient histological samples [6]. Likewise, epidemiological studies of the Japanese and Korean populations, which have continued to safely use probucol for its anti-atherosclerotic activity, could reveal reduced incidence of PD.

Taken together, probucol 1) exerts its multifaceted effect by modulating PINK1/Parkin mediated disrupted mitophagy, 2) improves survival, and 3) delays the lipodystrophy and sensory neurodegeneration resembling spinocerebellar ataxia phenotype in CLEC16A^{ΔUBC} (KO) mice. We conclude that in patient populations harboring variants that result in CLEC16A hypofunction, drugs with modulatory effects on mitophagy/SOCS1-JAK-STAT signaling could compensate for the attenuated CLEC16A activity and present formidable candidates for targeted interventions. Thus, our mouse model and current results with probucol and results with SOCS1-JAK-STAT pathway interventions, serve as a valuable tool for the assessment of therapeutic interventions in patients with diseases attributed to variants in CLEC16A. The therapeutics impact multiple autoimmune diseases by modulating and rescuing the observed dysregulated mitophagy/autophagy.

### REFERENCES

1. Pandey, R., et al., The Autoimmune Disorder Susceptibility Gene CLEC16A Restrains NK Cell Function in YTS NK Cell Line and Clec16a Knockout Mice. Frontiers in Immunology, 2019.10(68).
2. Pandey, R., et al., JAK/STAT inhibitor therapy partially rescues the lipodystrophic autoimmune phenotype in Clec16a KO mice. Sci Rep, 2021. 11(1): p. 7372.
3. Hain, H. S., et al., Inducible knockout of Clec16a in mice results in sensory neurodegeneration. Sci Rep, 2021. 11(1): p. 9319.
4. Georgakopoulos, N. D., G. Wells, and M. Campanella, The pharmacological regulation of cellular mitophagy. Nat Chem Biol, 2017. 13(2): p. 136-146.
5. Pandey, R., et al., CLEC16A regulates splenocyte and NK cell function in part through MEK signaling. PLoS One, 2018. 13(9): p. e0203952.
6. Brekk, O. R., et al., Cell type-specific lipid storage changes in Parkinson's disease patient brains are recapitulated by experimental glycolipid disturbance. Proc Natl Acad Sci U S A, 2020. 117(44): p. 27646-27654.

### Example VI

### Probucol Dose Response for Clec16aΔUBC Phenotype Rescue

CLEC16A has been implicated in numerous autoimmune diseases, and its dysfunction leads to dysregulated mitophagy, severe weight loss, inflammatory responses, and progressive neurodegeneration in Clec16aΔUBC mice. Our findings support the role of dysregulated mitophagy, UPR and ER stress in sensing danger and its contribution to aberrant immune response in autoimmune and autoinflammatory diseases. This mouse model mimics human sensory ataxia, dystonia, and lipodystrophy. To address these issues, we tested the mitophagy enhancer, Probucol (lipid lowering drug) and Quercetin (ER stress inhibitor) on our whole body inducible Clec16a KO to rescue the phenotype caused by Clec16a hypofunction.

Our findings provide evidence that targeting mitophagy and ER Stress pathways in combination with interventions at the JAK-STAT pathway results in the phenotype rescue. While current treatments for many autoimmune and neurodegenerative diseases only address symptom management without impacting disease progression. This lack of disease-modifying options highlights a significant gap in available therapies.

### Probucol Treatment

A graphical representation of the treatment protocol is shown in FIG. 8. Probucol was purchased from Cayman Chemical (cat#15043) and was formulated in saline (0.9%NaCl) with 2% DMSO, 2.5% PEG 300 and 2.5% Tween 80. The solution was administered intraperitoneally (IP) daily for 16 days at three doses of 3.5 mg/kg, 10 mg/kg, and 50 mg/kg (FIG. 8C). Vehicle treated mice received 2% DMSO, 2.5% PEG 300 and 2.5% Tween 80 in saline. A fresh solution was made daily prior to the injections. All animals were sacrificed according to humane endpoint according to the approved IACUC protocol.

### Probucol (Late-Stage Mitophagy Enhancer) Delays Phenotype Progression In A Dose Dependent Manner.

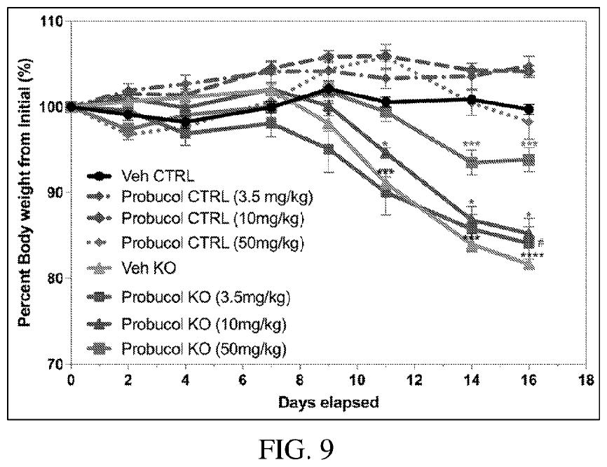

We conducted a comprehensive dose-response testing for probucol, carefully assessing its effects on the observed phenotype (FIG. 9). Our assessment involved gauging the effects of varying concentrations of probucol on the observable phenotype. As we progressively administered increasing doses of probucol (3.5 mg/kg, 10 mg/kg, and 50 mg/kg), a discernible trend emerged, revealing a more pronounced rescue of the phenotype (FIG. 9). Notable improvement was observable with the 10 mg/kg dosage by day 11. The 50 mg/kg of probucol displayed a robust rescue response as early as day 8 and maintained its significance throughout the study period. Probucol overall had a significant rescue effect on the organ weights of Clec16aΔUBC mice when compared to vehicle-treated-KO. Control tamoxifen, and probucol control littermates showed no adverse effect on organ weight or ratios over the course of the study. In general, Probucol at dosages of 3.5 mg/kg, 10 mg/kg, and 50 mg/kg exhibited a substantial and significant enhancement in the survival of Clec16a KO mice and a slowdown in the progression of the observed phenotype (FIG. 9).

This pattern indicates that higher doses of Probucol results in noteworthy enhancements in the phenotype under investigation. Evidently, there is a direct correlation between the degree of phenotype rescue and the dosage of probucol administered. This finding underscores the prospect of probucol as a compelling contender for ameliorating or even reversing the observed phenotype, further accentuating its potential relevance for therapeutic applications within our research context. This result reinforces the therapeutic potential of probucol as an effective enhancer of late stage mitophagy for the treatment of autoimmunity and neurodegeneration.

The positive relationship observed between probucol dosage, and the degree of phenotype rescue indicates that this compound holds promise for engaging with the specific biological mechanism under investigation. Our study delved deeply into the intricate intricacies of mitophagy rescue facilitated by probucol. Mitophagy, a critical process responsible for selectively clearing damaged mitochondria, holds paramount importance in preserving cellular well-being. Our experimental introduction of probucol yielded compelling results—a significant enhancement in the mitophagy process, aptly termed “mitophagy rescue.” This noteworthy revelation demonstrates probucol's capacity to amplify the removal of damaged mitochondria, a pivotal element in maintaining cellular function and forestalling the accumulation of flawed organelles, all of which would be beneficial in treating and/or preventing the propensity of autoimmunity and neurodegeneration in human.

Taken together, Probucol (late stage mitophagy enhancer) exerts its multifaceted effect by modulating PINK1/Parkin mediated disrupted mitophagy, improves survival, and delays the lipodystrophy and sensory neurodegeneration resembling spinocerebellar ataxia phenotype in Clec16aΔUBC (KO) mice in a dose dependent manner. We conclude that in patient populations harboring variants that result in CLEC16A hypofunction, drugs with modulatory effects on mitophagy/ER Stress/SOCS1-JAK-STAT signaling compensate for the attenuated CLEC16A activity and can be used in targeted interventions.

### Example VII

### Test and Treat Method for Ameliorating Symptoms Associated with CLEC16A Deficiency

The information herein above can be applied clinically to patients for therapeutic intervention, particularly for the treatment of symptoms associated with CLEC16A deficiency. A preferred embodiment of the invention comprises clinical application of the information described herein to a patient. In some embodiments, the CLEC16A-associated diseases or symptoms is assessed, monitored, or diagnosed by a method comprising: (i) measuring one or more clinical symptoms or signs of a CLEC16A deficiency in a subject, (ii) combining the measurements obtained into a single composite measurement, and (iii) assessing the overall severity of, or change in, the CLEC16A deficiency in the subject by comparing the composite measurement to a reference value or another composite measurement in the same subject. The (i) one or more composite measurements are employed to measure the clinical effect on the subject of a diagnostic, therapeutic or other type of medical intervention; (ii) for each of the measurements tested, the subject is classified as: (a) a responder or a non-responder, (b) a member of a clinical category, or (c) a member of a metric range, based on the change in said one or more clinical symptoms as measured using the particular clinical symptom or metabolic pathway assessed; and (iii) the measurements obtained are combined into a single composite measurement, by either: (a) separately assessing the change in each measurement obtained from each assay conducted prior to combining each measurement into a single composite measurement, or (b) combining measurements obtained from a first time point and generating a single composite measurement for said first time point and then comparing the single composite measurement for the first time point to a single composite measurement generated from the same assay for a second time point.

Important clinical assessments for CLEC16A-associated diseases or symptoms include amelioration or delayed progression of one or more of robust autoimmune inflammatory responses, severe weight loss, severe neurological symptoms, neuroinflammation, progressive neurodegeneration resembling spinocerebellar ataxia, and fat loss. Other clinical assessments include, enhanced clearance of mitochondria, the rescue spleen atrophy and/or improvements in the organ weight ratio of Thymus, inguinal white adipose tissue (iWAT), and/or gonal white adipose tissue (gWAT) when compared to an untreated control.

The derived therapeutic dose of mitophagy enhancer for human could be by those skilled in the art based on response rate. The mitophagy enhancer, or pharmaceutically acceptable composition comprising said mitophagy enhancer, can be administered at a dose of 0.0001 mg/kg/day, 0.001 mg/kg/day or 0.01 mg/kg/day to about 100 mg/kg/day, but may be higher or lower, depending upon, among other factors, the activity of the active metabolite compound, the bioavailability of the compound, its metabolism kinetics and other pharmacokinetic properties, the mode of administration and various other factors, discussed above.

Generally, the initial therapeutically effective amount of a compound described herein or a pharmaceutically acceptable salt thereof that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of the compound or a pharmaceutically acceptable salt thereof. In another embodiment, such dosage forms contain from about 50 mg to about 500 mg of the compound or a pharmaceutically acceptable salt thereof. In yet another embodiment, such dosage forms contain from about 25 mg to about 200 mg of the compound or a pharmaceutically acceptable salt thereof. In still another embodiment, such dosage forms contain from about 10 mg to about 100 mg of the compound or a pharmaceutically acceptable salt thereof. In a further embodiment, such dosage forms contain from about 5 mg to about 50 mg of the compound or a pharmaceutically acceptable salt thereof.

In certain embodiments, the mitophagy enhancer can be administered with one or more agents selected from a JAK-STAT inhibitor, an ER stress modulator, and a SOCS1 inhibitor. Exemplary JAK-STAT inhibitors include, without limitation, tofacitinib, ruxolitinib, baricitinib, peficitinib, decernotiniba, filgotinib, solcitinibb, itacitinib, SHR0302, upadacitinib, and PF-04965842. Exemplary ER stress modulators include, without limitation, Rapamycin, 4-phenylbutyric acid (4-PBA), trimethylamine N-oxide dehydrate (TMAO), dimethyl sulfoxide (DMSO), tauroursodeoxycholic acid (TUDCA), AMPK-activated protein kinase, 5′-aminoimidazole-4-carboxymide-1-β-d-ribofuranoside (AICAR), Glucagon-Like Peptide-1 (GLP-1), DPP4 inhibitors, and N-acetylcysteine (NAC).

Treatment can occur after a patient arrives in the clinic and presents with CLEC16A-associated diseases or symptoms. Mitophagy enhancers, such as probucol, quercetin, and acipimox, have been shown to be well tolerated and the symptoms were assessed using clinical scores criteria.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1-4. (Canceled)

5. A method for treating a CLEC16A-associated disorder or ameliorating at least one CLEC16A-associated symptom in a subject in need thereof, comprising administering an effective amount of at least one mitophagy enhancer, wherein said at least one symptom is selected from robust autoimmune inflammatory responses, severe weight loss, severe neurological symptoms, neuroinflammation, progressive neurodegeneration resembling spinocerebellar ataxia, and fat loss, wherein the mitophagy enhancer is selected from probucol, quercetin, or acipimox.

6. The method of claim 5, wherein the mitophagy enhancer is probucol.

7-10. (canceled)

11. The method of claim 5, wherein
a) the treatment with the mitophagy enhancer rescues spleen atrophy and/or improves the organ weight ratio of Thymus, inguinal white adipose tissue (iWAT), and/or gonal white adipose tissue (gWAT) when compared to an untreated control, or
b) the treatment delays CLEC16A-associated symptom progression when compared to an untreated control.

12. (canceled)

13. A method for treating CLEC16A-associated degeneration of an organ, comprising administration of a mitophagy enhancer, thereby altering the weight ratio in thymus and ameliorating symptoms associated with degeneration of the organ.

14. The method of claim 13, wherein the organ is the thymus or the spleen.

15. A method for treating CLEC16A-associated degeneration of white adipose tissue, comprising administration of a mitophagy enhancer, thereby altering the weight ratio in the white adipose tissue and ameliorating symptoms associated with degeneration of the white adipose tissue.

16. The method of claim 15, wherein the white adipose tissue is inguinal white adipose tissue (iWAT) or gonal white adipose tissue (gWAT).

17. The method of claim 1, further comprising administration of a PPARγ inhibitor.

18. (canceled)

19. The method of claim 13, wherein the CLEC16A-associated disorder is selected from an autoimmune disorder, a lipodystrophic disorder, and a neurodegenerative disorder.

20. The method of claim 13, wherein the at least one mitophagy enhancer enhances the clearance of mitochondria.

21. The method of claim 13, wherein the mitophagy enhancer is selected from probucol, quercetin, or acipimox.

22. (canceled)

23. The method of claim 13, further comprising administering one or more agents selected from a JAK-STAT inhibitor, an ER stress modulator, and a SOCS1 inhibitor.

24. The method of claim 23, wherein the agent is a JAK-STAT inhibitor selected from tofacitinib, ruxolitinib, baricitinib, peficitinib, decernotiniba, filgotinib, solcitinibb, itacitinib, SHR0302, upadacitinib, and PF-04965842, or the agent is an ER stress modulator selected from Rapamycin, 4-phenylbutyric acid (4-PBA), trimethylamine N-oxide dehydrate (TMAO), dimethyl sulfoxide (DMSO), tauroursodeoxycholic acid (TUDCA), AMPK-activated protein kinase, 5′-aminoimidazole-4-carboxymide-1-β-d-ribofuranoside (AICAR), Glucagon-Like Peptide-1 (GLP-1), DPP4 inhibitors, and N-acetylcysteine (NAC).

25-26. (canceled)

27. The method of claim 13, wherein the treatment with the mitophagy enhancer rescues spleen atrophy and/or improves the organ weight ratio of Thymus, inguinal white adipose tissue (iWAT), and/or gonal white adipose tissue (gWAT) when compared to an untreated control.

28. The method of claim 13, wherein the treatment delays CLEC16A-associated symptom progression when compared to an untreated control.
